(19) United States
(12) Patent Application Publication
(10) Pub. No.: US 2026/0085068 A1
(43) Pub. Date: Mar. 26, 2026

(54) A NOVEL CRYSTALLINE FORM OF PRITELIVIR

(71) Applicant: Assembly Biosciences, Inc., South San Francisco, CA (US)

(72) Inventors: Zhixin ZONG, South San Francisco, CA (US); Nicole WHITE, South San Francisco, CA (US); Yi WU, South San Francisco, CA (US)

(73) Assignee: Assembly Biosciences, Inc., South San Francisco, CA (US)

(21) Appl. No.: 19/106,358

(22) PCT Filed: Aug. 28, 2023
(86) PCT No.: PCT/IB2023/058487

§ 371 Date: Feb. 25, 2025

Related U.S. Application Data
(60) Provisional application No. 63/373,745, filed on Aug. 29, 2022.

(51) Int. Cl. C07D 417/12; A61K 31/4439

(52) U.S. Cl. CPC C07D 417/12; A61K 31/4439

(57) ABSTRACT

The invention relates to a novel crystalline form of pritelivir, as well as to pharmaceutical compositions comprising the same, and to methods for its production and use of the crystalline form in a medicament and for the treatment of herpes virus.

### FIELD OF INVENTION

The invention relates to a novel crystalline form of pritelivir, as well as to pharmaceutical compositions comprising the same, and to methods for its production and use of the crystalline form in a medicament and for the treatment of herpes virus.

### BACKGROUND OF INVENTION

Human herpes viruses are large-enveloped double-stranded DNA viruses that share the characteristic of establishing life-long infections in humans. This is accomplished by their ability to exist in the host either as a symptom free latent infection, where the virus lies dormant or, following activation, as a lytic infection with associated symptoms. These viral infections have widespread, worldwide prevalence and it is notable that over 90% of all humans are chronically infected with more than one human herpes virus.

Human herpes viruses are classified into three subfamilies (a, B and y) based upon their biological characteristics and the family consists of eight members, i.e., Herpes Simplex Virus subtype type 1 and 2 (HSV1, HSV2), Varicella Zoster Virus (VZV), Epstein-Barr virus (EBV), Cytomegalovirus (CMV), and Human Herpes Viruses 6-8 (HHV 6-8).

HSV1 and 2 infections can cause disease in immune competent individuals. Both subtypes cause cutaneous genital/anal and oro-labial/nasal cavity (cold sore) lesions, although HSV2 is more commonly associated with the former and HSV1 the latter. It is believed that >80% of genital infections are caused by HSV2. Globally, over 500 million people have genital herpes infections and approximately 50 to 80% of the world's population have oro-labial HSV infection, which is the main cause of cold sores. HSV, and particularly HSV1, can also cause lesions on the fingers (Whitlows) and other areas of the skin.

The vast majority of HSV infected individuals will not experience any noticeable symptoms. However, some will experience recurrent (and often severe) outbreaks of infection. In the USA, 20 to 40% of the population will get recurrent labial HSV lesions. Significantly, oro-labial cold sores and Whitlow's provide a very easy route for transmission of the virus to other individuals which can lead to rarer but much more serious HSV-related pathologies. For example, HSV-related ocular keratitis is a major cause of blindness and HSV can also cause encephalitis in neonates, which is a life-threatening condition. Other disorders believed to be caused by HSV include herpes gladiatorum, Mollaret's meningitis and possibly Bell's palsy.

Primary infection with, or reactivation of an existing herpes virus infection, can be a major cause of disease in immunocompromised individuals. Key at-risk populations include patients undergoing solid organ or stem cell transplantation, patients undergoing cancer treatment, individuals with HIV/AIDS, and ICU patients.

Presently, there is no cure for HSV. Medicines have been developed that can to some degree reduce the occurrence and/or shorten the length of outbreaks, but there is a need for improved therapies.

Currently, nucleoside analogues, such as acyclovir and its prodrugs, e.g., valacyclovir and famciclovir, are used as agents against herpes viruses such as HSV. In order to exert their effects, these nucleoside analogues must be phosphorylated by viral thymidine kinase (TK) and subsequently converted by cellular kinases to the nucleoside triphosphate, which inhibits the activity of the viral DNA polymerase. If the virus has no functionally active TK, as is the case, for example, with resistant HHV1 mutants or with TK-negative viruses, the nucleoside analogues are unable to exert their effects.

Nucleoside analogues are clinically administered at very high doses, e.g., doses as high as several hundred milligrams to several grams are typically administered per day. Even at these high doses, which are often administered over long treatment durations, these drugs are unable to completely prevent recurrent outbreaks of symptoms from HSV infection. Nucleoside analogues also do little to address the issue of viral shedding, which can asymptomatically facilitate the transmission of HSV to more individuals. Certain nucleoside analogues, particularly when used ay high doses, also give rise to safety concerns. For example, since these agents can incorporate into the genome DNA of a host via the host DNA polymerase, their mutagenicity is of concern, as documented for the nucleoside analogue, ganciclovir (Aoki, Chapter 45 in Mandell, Douglas and Bennett's Principles and Practice of Infectious Diseases (Eighth Edition) 2015).

Given the inadequacy of existing treatments, there is an urgent medical need to develop improved, well-tolerated anti-herpes treatments. One class of compounds that are currently being investigated are the helicase-primase inhibitors. Helicase-primase inhibitors are antiviral agents with a novel mechanism of action. They inhibit the viral heterotrimeric complex consisting of helicase, primase, and cofactor subunits, which have functions that are essential for viral DNA replication. These agents are not nucleoside analogues and do not require phosphorylation by TK to inhibit HSV replication. They are therefore potentially active against TK-deficient HSV, which as described above, is a major mechanism of resistance to nucleoside analogues.

One example of a helicase-primase inhibitor is pritelivir, a thiazolylamide derivative with the chemical name N-Methyl-N-(4-methyl-5-sulfamoyl-1,3-thiazol-2-yl)-2-[4-(pyridin-2-yl)phenyl]acetamide. The compound has been disclosed in WO 2001/47904.

WO 2006/103011 generally mentions that pritelivir free base may exist in hydrated or solvated forms; however it does not actually disclose any experimental details regarding preparation or characterization of these forms. WO 2013/045491 and WO 2013/045479 concern a mesylate salt of pritelivir. They both mention that there are polymorphic and solvated forms of the free base but neither publication actually experimentally disclose these forms. WO 2013/045491 concludes that the free base of pritelivir is not suitable for a long-term stable formulation and the low solubility of the free base is responsible for unfavourable drug release and resorption properties compared to a mesylate mono-hydrate salt, while WO 2013/045497 concludes that the free base has low thermal and polymorphic stability and is unsuitable for tableting. Both WO 2013/045491 and WO 2013/045479 disclose that the mesylate salt is especially well suited for oral compositions.

WO 2018/096170 discloses a polymorphic screen of pritelivir free base and it is mentioned that 14 different forms were observed as well as an additional form during the scale-up phase. The majority of these forms were solvated forms; only 3 hydrates and one anhydrous form were observed. The clear focus of WO 2018/096170 is Form C (a hemihydrate). This is the only form for which XRPD characterization data is provided. WO 2018/096170 states that the free base hemihydrate Form C was the only stable polymorph. The present invention was devised with the foregoing in mind.

### SUMMARY OF THE INVENTION

Provided herein is a novel crystalline solid form of pritelivir useful for use in a medicament. Pritelivir has the following structural formula:

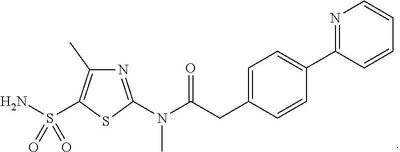 [SMILES: Cc1nc(N(C)C(=O)Cc2ccc(-c3ccccn3)cc2)sc1S(N)(=O)=O]

The present disclosure relates to a certain novel solid form of pritelivir which possesses promising and advantageous solid-state and/or biopharmaceutical properties.

In a first aspect, the present disclosure provides a crystalline form of pritelivir, wherein the crystalline form is Type 8 and is characterized by data selected from one or more of the following:
a) an X-ray powder diffraction (XRPD) pattern measured using Cu Ka (λ=1.5406 Å) comprising peaks at 2-theta values of 10.5 and 24.7°2θ±0.2°2θ; and
b) a DSC thermogram comprising an endothermic event with an onset temperature of 224° C.±2° C.

Type 8 is a non-solvated and anhydrous crystalline form of pritelivir free base and has a high melting point. A high melting point means that Type 8 can be handled at high temperatures without it melting, for example, during size reduction procedures, i.e., micronization and the manufacturing of a pharmaceutical product.

Advantageously, Type 8 has certain characteristics that make it particularly suited to formulation and delivery via certain routes of administration. The solubility of Type 8 at room temperature in aqueous media containing 0.9% NaCl and 0.5% polysorbate 80 at 24 hours was determined to be around 70.7 μg/mL (see Example 2 of the present application).

In another aspect, the present disclosure provides a pharmaceutical composition comprising a crystalline form of pritelivir wherein the crystalline form is Type 8 and a pharmaceutically acceptable carrier, diluent or excipient.

In a further aspect, the present invention provides a method of treating a herpes virus (conveniently a HSV) infection in a subject in need thereof, the method comprising administering to the subject a therapeutically effective amount of a crystalline form of pritelivir, wherein the crystalline form is Type 8.

In a further aspect, the present invention provides a method of treating a herpes virus (conveniently a HSV) infection in a subject in need thereof, the method comprising administering to the subject a therapeutically effective amount of a pharmaceutical composition comprising a crystalline form of pritelivir and a pharmaceutically acceptable carrier, diluent or excipient, wherein the crystalline form is Type 8.

In another aspect, there is provided a crystalline form of pritelivir for use in the treatment of a herpes virus (conveniently a HSV) infection, wherein the crystalline form is Type 8.

In another aspect, there is provided a pharmaceutical composition comprising a crystalline form of pritelivir, and a pharmaceutically acceptable carrier, diluent or excipient for use in the treatment of a herpes virus (conveniently a HSV) infection, wherein the crystalline form is Type 8.

In another aspect, there is provided the use of a crystalline form of pritelivir or the use of a pharmaceutical composition comprising pritelivir for the treatment of a herpes virus (conveniently a HSV) infection, wherein the crystalline form is Type 8.

In another aspect, there is provided the use of a crystalline form of pritelivir or a pharmaceutical composition comprising a crystalline form of pritelivir in the manufacture of a medicament for treating a herpes virus (conveniently a HSV) infection, wherein the crystalline form is Type 8.

In another aspect, there is provided a process to prepare a crystalline form of pritelivir wherein the crystalline form is Type 8.

In another aspect, there is provided a crystalline form of pritelivir obtainable by the process described herein, wherein the crystalline form is Type 8.

### BRIEF DESCRIPTION OF THE DRAWINGS

The summary, as well as the following detailed description, is further understood when read in conjunction with the appended drawings. For the purpose of illustrating the disclosed compositions and methods, there are shown in the drawings exemplary embodiments of the compositions and methods; however, the compositions and methods are not limited to the specific embodiments disclosed. In the drawings:

FIG. 1: XRPD diffractogram of Type 8.

FIG. 2: DSC and TGA thermograms of Type 8.

FIG. 3: XRPD diffractogram of Form C.

### DETAILED DESCRIPTION

The features and other details of the disclosure will now be more particularly described. Before further description of the present disclosure is provided, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

Unless otherwise stated, the following terms used in the specification and claims have the following meanings set out below.

As used herein, “pritelivir” refers to N-methyl-N-(4-methyl-5-sulfamoylthiazol-2-yl)-2-(4-(pyridin-2-yl)phenyl) acetamide free base.

As used herein, “API” refers to an active pharmaceutical ingredient, e.g., pritelivir.

Unless the context requires otherwise, throughout this specification and claims, the words “comprise,” “comprising” and the like are to be construed in an open, inclusive sense; and the words “a,” “an,” and the like are to be considered as meaning at least one and are not limited to just one. Terms not specifically defined herein should be given the meanings that would be given to them by one of skill in the art in light of the disclosure and the context.

When values are expressed as approximations, by use of the antecedent “about,” it will be understood that the particular value forms another embodiment. Further, the term “about” refers to a ±10% variation from the nominal value unless otherwise indicated or inferred.

The term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

As used herein, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

The use of any and all examples, or exemplary language herein, for example, “such as,” “including,” or “for example,” is intended merely to illustrate better the present teachings and does not pose a limitation on the scope of the invention unless claimed. No language in the specification should be construed as indicating any non-claimed element as essential to the practice of the present teachings.

The terms “individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, dogs, primates, and the like). The mammal treated in the methods of the disclosure is desirably a mammal in which treatment of HSV infection is desired.

The term “modulation” includes antagonism (e.g., inhibition), agonism, partial antagonism and/or partial agonism.

The term “pharmaceutically acceptable” include molecular entities and compositions that do not produce an adverse, allergic or other untoward reaction when administered to an animal, or a human, as appropriate.

The term “pharmaceutically acceptable carrier” or “pharmaceutically acceptable excipient” as used herein refers to any and all solvents, dispersion media, coatings, fillers, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carrier, diluent or excipient.

The term “therapeutically effective amount” or “effective amount” as used herein refers to the amount of the subject compound that will elicit the biological or medical response of a tissue, system or animal, (e.g., mammal or human) that is being sought by the researcher, veterinarian, medical doctor or other clinician. The compounds or pharmaceutical compositions of the disclosure are administered in therapeutically effective amounts to treat a disease. Alternatively, a therapeutically effective amount of a compound is the quantity required to achieve a desired therapeutic and/or prophylactic effect. The therapeutically effective dose may vary according to factors such as the disease state, age, sex, and weight of the individual, and the ability of the composition to cause a desired response in a subject. Such results include, but are not limited to, the reduction, remission, and/or regression of conditions caused by, or associated with, HSV or prevention of the development of conditions caused by, or associated with, HSV, as determined by any means suitable in the art.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms. As used herein, “treating” and like terms may specifically include reducing the severity and/or frequency of HSV induced symptoms, eliminating HSV induced symptoms and/or the underlying cause of said symptoms, reducing the frequency or likelihood of HSV induced symptoms and/or their underlying cause, delaying, preventing and/or slowing the progression of HSV induced conditions, and improving or remediating damage caused, directly or indirectly, by HSV infections.

The term “preventing,” as used herein with respect to an HSV infection or HSV-related disorder, refers to reducing the likelihood of HSV infection.

Reference to a particular numerical value includes at least that particular value, unless the context clearly dictates otherwise.

At various places in the present specification, values are disclosed in groups or in ranges. It is specifically intended that the description include all individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. All ranges are inclusive and combinable.

The use of any and all examples, or exemplary language herein, for example, “such as,” “including,” or “for example,” is intended merely to illustrate better the present teachings and does not pose a limitation on the scope of the invention unless claimed.

A “crystalline form” is a solid material wherein the constituents of the solid material are arranged in a highly ordered microscopic structure, thereby forming a crystal lattice which extends in all directions. Crystalline forms can include anhydrous crystalline forms, solvated crystalline forms and/or hydrated crystalline forms.

“Polymorphism” is when a solid material can exist in more than one crystalline form.

As used herein, the term “amorphous” refers to a solid material having no long-range order in the position of its molecules. Amorphous solids are substances in which the molecules are arranged in a random manner so that there is no well-defined arrangement, e.g., molecular packing, and no long-range order. Amorphous solids are generally isotropic, i.e., exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid.

A “hydrate” is a compound that exists in a solid composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein. Hydrates may be crystalline, wherein both the compound and water form part of the crystal lattice.

A “solvate” is a similar composition to a hydrate except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein. Solvates may be crystalline, wherein both the compound and solvent form part of the crystal lattice.

“Anhydrous” means the solid form of the compound does not have water incorporated into its structure. For example, an anhydrous crystalline form does not have water forming part of the crystal structure. The skilled person would be aware of techniques which can be used to quantify the amount of water associated with a solid. For example, water content can be determined by either Karl Fischer Titration or Thermogravimetric Analysis (TGA). Suitably, an anhydrous solid form of the compound comprises less than about 1.5% by weight, such as less than about 1, less than about 0.5, less than about 0.4, less than about 0.3, less than about 0.2, less than about 0.1, less than about 0.05, or less than about 0.01% by weight of water.

“Un-solvated” or “non-solvated” means the solid form of the compound does not have solvent(s) incorporated into its structure. For example, an un-solvated crystalline form does not have solvent(s) forming part of the crystal structure. The skilled person would be aware of techniques which can quantify the amount of solvent associated with a solid. For example, solvent content can be determined by Gas Chromatography (GC). Suitably, an un-solvated or non-solvated solid form of the compound comprises less than about 1.5% by weight, such as less than about 1.0, less than about 0.5, less than about 0.4, less than about less than 0.3, less than about 0.2, less about than 0.1, less than about 0.05, or less than about 0.01% by weight of solvent.

Herein, where a composition is said to “consist essentially of” a particular component, said composition suitably comprises at least 70 wt % of said component, suitably at least 80 wt % thereof, suitably at least 90 wt % thereof, suitably at least 95 wt % thereof, most suitably at least 99 wt % thereof. Suitably, a composition said to “consist essentially of” a particular component consists of said component save for one or more trace components.

The phrase “substantially as shown in Figure” refers to an X-ray powder diffraction pattern or DSC thermogram with at least 50%, or at least 60%, or at least 70%, or at least 80%, or at least 90%, or at least 95% or at least 99% of its features appearing in the Figure.

### Crystalline Form of Pritelivir

In one aspect, the present disclosure provides a novel crystalline form of pritelivir described and characterized herein as Type 8.

The present disclosure is also directed to pharmaceutical compositions comprising the crystalline form Type 8, and to methods for preparing such form. The present disclosure is further directed to the use of the crystalline form Type 8 in the treatment or prevention of HSV infections.

There are a number of analytical methods one of ordinary skill in the art in solid-state chemistry can use to analyze solid forms. The term “analyze” as used herein means to obtain information about the solid-state structure of solid forms. For example, powder X-ray diffraction (PXRD/XRPD) is a suitable technique for differentiating amorphous solid forms from crystalline solid forms and for characterizing and identifying particular crystalline solid forms of a compound.

Due to differences in instruments, samples, and sample preparation, peak values are often reported with the modifier “+0.2°20”. This is common practice in the solid-state chemical arts because of the variation inherent in peak values. Variability in peak intensity is a result of how individual crystals are oriented in the sample container with respect to the external X-ray source (known as “preferred orientation”). This orientation effect does not provide structural information about the crystal.

Powder X-ray diffraction is just one of several analytical techniques one may use to characterize and/or identify crystalline solid forms. Differential scanning calorimetry (DSC) may be used to characterize and/or identity crystalline solid forms. A typical variability for a value associated with a differential scanning calorimetry onset temperature is on the order of plus or minus 2° C.

It should be noted that unless noted otherwise, thermal data (DSC and TGA) presented herein were acquired using a heating rate of 10° C./min. Furthermore, DSC data was acquired using Aluminum punched pans.

In a first aspect, there is provided a crystalline form of pritelivir wherein the crystalline form is Type 8 and is characterized by data selected from one or more of the following:
a) an X-ray powder diffraction (XRPD) pattern measured using Cu Ka (λ=1.5406 Å) comprising peaks at 2-theta values of 10.5 and 24.7°2θ±0.2°2θ; and
b) a DSC thermogram comprising an endothermic event with an onset temperature of 224° C.±2° C.

In one embodiment, there is provided an XRPD pattern measured using Cu Ka (λ=1.5406 Å) comprising peaks at 2-theta values of 10.5 and 24.7°2θ±0.2°2θ and further comprising at least one, two, three or four specific peaks selected from peaks at 2-theta values of 17.4, 17.9, 23.0 and 23.3°2θ±0.2°2θ.

In one embodiment, there is provided an XRPD pattern measured using Cu Ka (λ=1.5406 Å) comprising peaks at 2-theta values of 10.5 and 24.7°2θ±0.2°2θ and further comprising peaks at 2-theta values of 17.4, 17.9, 23.0 and 23.3°2θ±0.2°2θ.

In one embodiment, there is provided an XRPD pattern measured using Cu Ka (λ=1.5406 Å) comprising peaks at 2-theta values of 10.5 and 24.7°2θ±0.1°2θ.

In one embodiment, there is provided an XRPD pattern measured using Cu Ka (λ=1.5406 Å) comprising peaks at 2-theta values of 10.5 and 24.7°2θ±0.1°2θ and further comprising at least one, two, three or four specific peaks selected from peaks at 2-theta values of 17.4, 17.9, 23.0 and 23.3°2θ±0.1°2θ.

In one embodiment, there is provided an XRPD pattern measured using Cu Ka (λ=1.5406 Å) comprising peaks at 2-theta values of 10.5 and 24.7°2θ±0.1°2θ and further comprising peaks at 2-theta values of 17.4, 17.9, 23.0 and 23.3°2θ±0.1°2θ.

In one embodiment, there is provided an XRPD pattern measured using Cu Ka (λ=1.5406 Å) comprising peaks at 2-theta values of 10.5 and 24.7°2θ±0.2°2θ, suitably 10.5 and 24.7°2θ±0.1°2θ, and wherein Type 8 is un-solvated and anhydrous. Suitably, Type 8 comprises less than about 2% by weight solvent and/or water, such as less than about 1.5% by weight, less than about 1%, less than about 0.5, less than about 0.4, less than about 0.3, less than about 0.2 or less than about 0.1% by weight.

In one embodiment, there is provided a crystalline form of a Compound of Formula (I) wherein the crystalline form is Type 8 characterized by an XRPD pattern measured using Cu Ka (λ=1.5406 Å) comprising peaks at 2-theta values of 10.5 and 24.7°2θ±0.2°2θ and further comprising at least two, five, ten, fifteen, twenty, twenty-five, thirty or thirty-five further peaks selected from the group consisting of the peaks in Table 1 in ° 2θ±0.2°2θ.

TABLE 1
XRPD peak positions for Type 8
Peak
Position
°2θ

9.2
10.3
13.7
13.9
14.9
15.4
16.3
17.3
17.4
17.9
18.7
20.9
21.4
22.0
23.0
23.3
23.9
25.1
25.5
25.8
26.1
27.4
28.2
28.8
29.6
29.9
30.2
30.5
32.9
33.2
33.7
34.9
35.5
36.8
38.1
38.9

In one embodiment, there is there is provided a crystalline form of a Compound of Formula (I) wherein the crystalline form is Type 8 characterized by an XRPD pattern measured using Cu Ka (λ=1.5406 Å) comprising peaks at 2-theta values of 10.5 and 24.7°2θ±0.1°2θ; and further comprising at least two, five, ten, fifteen, twenty, twenty-five, thirty or thirty-five peaks selected from the group consisting of the peaks in Table 1 in °2θ±0.1°2θ.

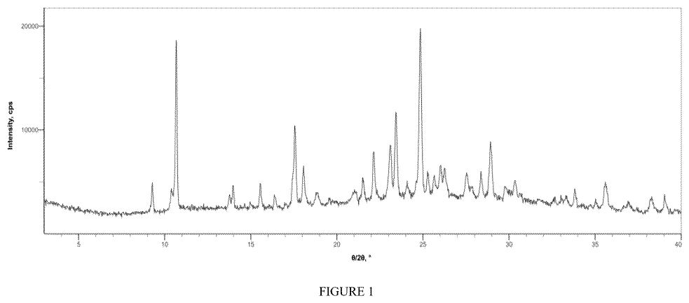

In one embodiment, there is provided a crystalline form of pritelivir wherein the crystalline form is Type 8 characterized by an XRPD pattern measured using Cu Ka (λ=1.5406 Å) radiation substantially the same as shown in FIG. 1.

In one embodiment, there is provided a crystalline form of pritelivir wherein the crystalline form is Type 8 characterized by a DSC thermogram comprising an endothermic event with an onset temperature of 224° C.±2° C., suitably 224° C.±1° C.

In one embodiment, there is provided a crystalline form of pritelivir wherein the crystalline form is Type 8 characterized by a DSC thermogram comprising an endothermic event with an onset temperature of 224° C.±2° C., suitably 224° C.±1° C., and wherein Type 8 is anhydrous and un-solvated.

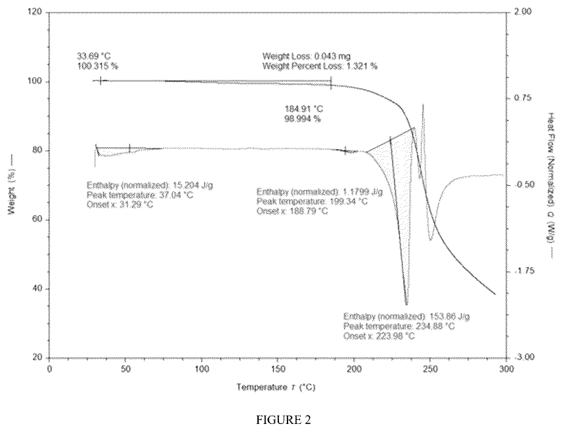

In one embodiment, there is provided a crystalline form of pritelivir wherein the crystalline form is Type 8 characterized by a DSC thermogram substantially the same as FIG. 2.

In one embodiment, Type 8 is substantially pure.

In one embodiment, Type 8 comprises less than about 2% by weight solvent and/or water, such as less than about 1.5% by weight, less than about 1%, less than about 0.5%, less than about 0.4%, less than about 0.3%, less than about 0.2%, or less than about 0.1% by weight. The skilled person would know of suitable analytical techniques which can quantify the amount of solvent/water associated with a solid. For example, water content can be determined by Karl Fischer Titration. Residual solvents can be determined by Gas Chromatography. Thermogravimetric Analysis (TGA) can also quantify the amount of volatile material (i.e., solvent and water) associated with a solid (either surface bound or incorporated into the crystal structure). Suitably, Type 8 is anhydrous and un-solvated.

When it is stated herein that the specification relates to a crystalline form of pritelivir, the degree of crystallinity is conveniently greater than about 60%, more conveniently greater than about 80%, yet more conveniently greater than about 90% and preferably greater than 95%, 98% or 99% by weight.

In one embodiment, Type 8 is pure or substantially pure. As used herein, the term “substantially pure” means that the solid state form of pritelivir contains about 20% by weight or less, or about 15% by weight or less, or about 10% by weight or less, or about 5% by weight or less, or about 2% by weight or less, or about 1% by weight or less, or about 0.5 by weight or less of any impurities or other solid forms of pritelivir, including alternative crystalline forms, hydrates, solvates or amorphous forms, for example as measured by XRPD. Thus, substantially pure Type 8 described herein would be understood to contain greater than about 80% by weight, greater than 85% by weight, greater than 90% by weight, greater than 95% by weight, greater than 98% by weight, greater than 99% by weight, greater than 99.5% by weight of crystalline Type 8 of pritelivir. Suitably, there is provided Type 8 wherein when Type 8 is analyzed by a solid-state technique, such as by X-Ray Powder diffraction, no other solid forms (amorphous and/or other crystalline forms) are detected. Suitably, there is provided a crystalline form of pritelivir essentially consisting of Type 8. Suitably, there is provided a crystalline form of pritelivir consisting of Type 8.

### Pharmaceutical Compositions and Kits

In another aspect, the present disclosure provides pharmaceutical compositions comprising a crystalline form of pritelivir as disclosed herein formulated together with one or more pharmaceutically acceptable carrier, diluent or excipient, wherein the crystalline form is Type 8.

In one embodiment, there is provided a pharmaceutical composition comprising a crystalline form of pritelivir wherein the crystalline form is Type 8; and a pharmaceutically acceptable carrier, diluent or excipient. In one embodiment, there is provided a pharmaceutical composition comprising a crystalline form of pritelivir wherein the crystalline form is pure or substantially pure Type 8; and a pharmaceutically acceptable carrier, diluent or excipient. In one embodiment, there is provided a pharmaceutical composition comprising pritelivir wherein pritelivir consists essentially of crystalline Type 8. In one embodiment, there is provided a pharmaceutical composition comprising pritelivir wherein pritelivir consists of crystalline Type 8.

When it is stated herein that the specification relates to a crystalline form of pritelivir, the degree of crystallinity is conveniently greater than about 60%, more conveniently greater than about 80%, yet more conveniently greater than about 90% and preferably greater than 95%, 98% or 99% by weight.

These formulations include those suitable for oral, rectal, topical, buccal, parenteral, rectal, vaginal, or aerosol administration, although the most suitable form of administration in any given case will depend on the degree and severity of the condition being treated and on the nature of the particular compound being used.

In one embodiment, there is provided a pharmaceutical composition comprising a crystalline form of pritelivir as disclosed herein, formulated together with one or more pharmaceutically acceptable carrier, diluent or excipient, wherein the crystalline form is Type 8, and wherein the composition is suitable for parenteral administration.

Exemplary pharmaceutical compositions of this disclosure may be used in the form of a pharmaceutical preparation, for example, in solid, semisolid or liquid form, which contains one or more compounds of the disclosure, as an active ingredient, in admixture with an organic or inorganic carrier or excipient suitable for external, enteral or parenteral applications. The active ingredient may be compounded, for example, with the non-toxic, pharmaceutically acceptable carriers for any form suitable for use. The active object compound is included in the pharmaceutical composition in an amount sufficient to produce the desired effect upon the process or condition of the disease.

Advantageously, the disclosure also provides kits for use by e.g., a consumer in need of HSV infection treatment. Such kits include a suitable dosage form such as those described above and instructions describing the method of using such dosage form to mediate, reduce or prevent HSV infection. The instructions would direct the consumer or medical personnel to administer the dosage form according to administration modes known to those skilled in the art. Such kits could advantageously be packaged and sold in single or multiple kit units. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . , etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### Methods of Treatment

In one aspect, there is provided crystalline pritelivir Type 8 for use as a medicament.

In one aspect, there is provided a pharmaceutical composition comprising a crystalline form of pritelivir and a pharmaceutically acceptable carrier, diluent or excipient, wherein the crystalline form is Type 8, for use as a medicament.

In one aspect, there is provided crystalline pritelivir Type 8 for use in therapy.

In one aspect, there is provided a pharmaceutical composition comprising a crystalline form of pritelivir and a pharmaceutically acceptable carrier, diluent or excipient, wherein the crystalline form is Type 8, for use in therapy.

In one aspect, there is provided a method for treating a herpes virus (conveniently a HSV) infection in a subject in need thereof, the method comprising administering to a subject a therapeutically effective amount of a crystalline form of pritelivir wherein the crystalline form is Type 8. In another aspect, there is provided a method for treating a herpes virus (conveniently a HSV) infection in a subject in need thereof is provided, comprising administering to a subject a therapeutically effective amount of a pharmaceutical composition comprising a crystalline form of pritelivir and a pharmaceutically acceptable carrier, diluent or excipient, wherein the crystalline form is Type 8.

In one embodiment, there is provided a method for inhibiting HSV replication in a subject in need thereof, the method comprising administering to a subject a therapeutically effective amount of a crystalline form of pritelivir wherein the crystalline form is Type 8. In one embodiment, there is provided a method for inhibiting HSV replication in a subject in need thereof is provided, comprising administering to a subject a therapeutically effective amount of a pharmaceutical composition comprising a crystalline form of pritelivir and a pharmaceutically acceptable carrier, diluent or excipient, wherein the crystalline form is Type 8.

In one embodiment, there is provided a method for reducing the likelihood or severity of symptoms of a HSV infection in a subject in need thereof, the method comprising administering to a subject a therapeutically effective amount of a crystalline form of pritelivir wherein the crystalline form is Type 8. In one embodiment, there is provided a method for reducing the likelihood or severity of symptoms of a HSV infection in a subject in need thereof is provided, comprising administering to a subject a therapeutically effective amount of a pharmaceutical composition comprising a crystalline form of pritelivir and a pharmaceutically acceptable carrier, diluent or excipient, wherein the crystalline form is Type 8.

In one embodiment, there is provided a method for inhibiting the development or progression of a disease or disorder caused by, or associated with, HSV infection, in a subject in need thereof, the method comprising administering to a subject a therapeutically effective amount of a crystalline form of pritelivir wherein the crystalline form is Type 8. In one embodiment, there is provided a method for inhibiting the development or progression of a disease or disorder caused by, or associated with, HSV infection, in a subject in need thereof is provided, comprising administering to a subject a therapeutically effective amount of a pharmaceutical composition comprising a crystalline form of pritelivir and a pharmaceutically acceptable carrier, diluent or excipient, wherein the crystalline form is Type 8.

In one embodiment, there is provided a method for treating or preventing a disease or disorder caused by, or associated with, HSV infection, in a subject in need thereof, the method comprising administering to a subject a therapeutically effective amount of a crystalline form of pritelivir wherein the crystalline form is Type 8. In one embodiment, there is provided a method for treating or preventing a disease or disorder caused by, or associated with, HSV infection, in a subject in need thereof is provided, comprising administering to a subject a therapeutically effective amount of a pharmaceutical composition comprising a crystalline form of pritelivir and a pharmaceutically acceptable carrier, diluent or excipient, wherein the crystalline form is Type 8.

In a particular embodiment, pritelivir Type 8 according to the present invention, or the pharmaceutical compositions according to the present invention can reduce time to healing of lesions (e.g., time to full recovery of lesions) and duration of symptoms resulting from HSV infections in diseases or disorders, such as herpes labialis or genital herpes. The time to lesion healing may be defined as complete epithelization of mucocutaneous HSV lesion(s) within the treatment period and no appearance of new lesions, e.g., as assessed by a physician.

In one embodiment, pritelivir Type 8 according to the present invention, or the pharmaceutical compositions according to the present invention can reduce pain or pain intensity (for example, at a lesion site) caused as a consequence of HSV infections in diseases or disorders, such as herpes labialis or genital herpes.

In another aspect, there is provided a crystalline form of pritelivir for use in the treatment or of a herpes virus (conveniently a HSV) infection, wherein the crystalline form is Type 8. In another aspect, there is provided a pharmaceutical composition comprising a crystalline form of pritelivir, and a pharmaceutically acceptable carrier, diluent or excipient for use in the treatment of a herpes virus (conveniently a HSV) infection, wherein the crystalline form is Type 8.

In one embodiment, there is provided a crystalline form of pritelivir for use in the inhibition of HSV replication in a subject, wherein the crystalline form is Type 8. In one embodiment, there is provided a pharmaceutical composition comprising a crystalline form of pritelivir, and a pharmaceutically acceptable carrier, diluent or excipient for use in the inhibition of HSV replication in a subject, wherein the crystalline form is Type 8.

In one embodiment, there is provided a crystalline form of pritelivir for use in reducing the likelihood or severity of symptoms of a HSV infection in a subject in need thereof, wherein the crystalline form is Type 8. In one embodiment, there is provided a pharmaceutical composition comprising a crystalline form of pritelivir, and a pharmaceutically acceptable carrier, diluent or excipient for use in reducing the likelihood or severity of symptoms of a HSV infection in a subject in need thereof, wherein the crystalline form is Type 8.

In one embodiment, there is provided a crystalline form of pritelivir for use in inhibiting the development or progression of a disease or disorder caused by, or associated with, HSV infection, in a subject in need thereof, wherein the crystalline form is Type 8. In one embodiment, there is provided a pharmaceutical composition comprising a crystalline form of pritelivir, and a pharmaceutically acceptable carrier, diluent or excipient for use in inhibiting the development or progression of a disease or disorder caused by, or associated with, HSV infection, in a subject in need thereof, wherein the crystalline form is Type 8.

In one embodiment, there is provided a crystalline form of pritelivir for use in treating or preventing a disease or disorder caused by, or associated with, HSV infection, in a subject in need thereof, wherein the crystalline form is Type 8. In one embodiment, there is provided a pharmaceutical composition comprising a crystalline form of pritelivir, and a pharmaceutically acceptable carrier, diluent or excipient for use in treating or preventing a disease or disorder caused by, or associated with, HSV infection, in a subject in need thereof, wherein the crystalline form is Type 8.

In another aspect, there is provided the use of a crystalline form of pritelivir or the use of a pharmaceutical composition comprising a crystalline form of pritelivir for the treatment of a herpes virus (conveniently a HSV) infection, wherein the crystalline form is Type 8.

In one embodiment, there is provided the use of a crystalline form of pritelivir or the use of a pharmaceutical composition comprising a crystalline form of pritelivir for the inhibition of HSV replication in a subject, wherein the crystalline form is Type 8.

In one embodiment, there is provided the use of a crystalline form of pritelivir or the use of a pharmaceutical composition comprising a crystalline form of pritelivir for use in reducing the likelihood or severity of symptoms of a HSV infection in a subject in need thereof, wherein the crystalline form is Type 8.

In one embodiment, there is provided the use of a crystalline form of pritelivir or the use of a pharmaceutical composition comprising a crystalline form of pritelivir for use in inhibiting the development or progression of a disease or disorder caused by, or associated with, HSV infection, in a subject in need thereof, wherein the crystalline form is Type 8.

In one embodiment, there is provided the use of a crystalline form of pritelivir or the use of a pharmaceutical composition comprising a crystalline form of pritelivir for use in treating or preventing a disease or disorder caused by, or associated with, HSV infection, wherein the crystalline form is Type 8.

In another aspect, there is provided the use of a crystalline form of pritelivir or the use of a pharmaceutical composition comprising a crystalline form of pritelivir in the manufacture of a medicament for treating a herpes virus (conveniently a HSV) infection, wherein the crystalline form is Type 8.

In one embodiment, there is provided the use of a crystalline form of pritelivir or the use of a pharmaceutical composition comprising a crystalline form of pritelivir in the manufacture of a medicament for inhibiting HSV replication in a subject, wherein the crystalline form is Type 8.

In one embodiment, there is provided the use of a crystalline form of pritelivir or the use of a pharmaceutical composition comprising a crystalline form of pritelivir in the manufacture of a medicament for reducing the likelihood or severity of symptoms of a HSV infection in a subject in need thereof, wherein the crystalline form is Type 8.

In one embodiment, there is provided the use of a crystalline form of pritelivir or the use of a pharmaceutical composition comprising a crystalline form of pritelivir in the manufacture of a medicament for inhibiting the development or progression of a disease or disorder caused by, or associated with, HSV infection, in a subject in need thereof, wherein the crystalline form is Type 8.

In one embodiment, there is provided the use of a crystalline form of pritelivir or the use of a pharmaceutical composition comprising a crystalline form of pritelivir in the manufacture of a medicament for treating or preventing a disease or disorder caused by, or associated with, HSV infection, in a subject in need thereof, wherein the crystalline form is Type 8.

In a particular embodiment, the disease or disorder caused by, or associated with, HSV infection, is selected from herpes labialis (e.g., oro-labial cold sores or Whitlow's), genital herpes, HSV-related ocular keratitis, HSV-related encephalitis, herpes gladiatorum, primary HSV gingivostomatitis, Mollaret's meningitis and Bell's palsy.

In a particular embodiment, the disease or disorder caused by, or associated with, HSV infection, is selected from herpes labialis (e.g., oro-labial cold sores or Whitlow's) or genital herpes. In one embodiment, the disease or disorder is recurrent herpes labialis or recurrent genital herpes. Individuals with a history of multiple recurrences of herpes labialis or recurrent genital herpes, e.g., HSV which recurs six times or more annually, may be regarded as having recurrent HSV.

In one embodiment, the herpes virus being treated is HSV2. In a further embodiment, the herpes virus being treated is HSV2 and the subject in need of the treatment has HSV2 recurrent genital herpes.

In one embodiment, the herpes virus being treated is HSV1. In yet a further embodiment, both herpes virus HSV-1 and HSV-2 are being treated.

In one embodiment, the herpes virus being treated or prevented is resistant to nucleosidic anti-viral therapy. In one embodiment, the nucleosidic antiviral therapy is selected from the group consisting of acyclovir, penciclovir, famciclovir, ganciclovir and valacyclovir.

In one embodiment, the herpes virus being treated is resistant to nucleosidic antiviral therapy, e.g., acyclovir-resistant mucocutaneous HSV infection. In a further embodiment, the HSV infection being treated is a mucocutaneous HSV infection resistant to therapy with antiviral therapy with nucleoside analogues, such as acyclovir, penciclovir, famciclovir, ganciclovir or valacyclovir.

In a particular embodiment, the subject in need of the methods disclosed herein, is immunocompromised. The subject may be immunocompromised due to conditions including HIV infection, cancer, hematopoietic cell or solid organ transplantation and chronic glucocorticoid use or a genetic immunodeficiency.

In a particular embodiment, the subject in need of the methods disclosed herein, is a neonate or an infant.

In a particular embodiment, the subject is a herpes-positive patient.

In a particular embodiment, the subject in need of the methods disclosed herein, has acyclovir-resistant mucocutaneous HSV infection. This subject may have been diagnosed with condition on the basis of clinical failure, e.g., no improvement after oral or iv doses for at least 7 days with approved doses of acyclovir.

In a particular embodiment, the subject in need of the methods disclosed herein, has a primary genital HSV-related herpes infection. In one aspect, the subject in need of the methods disclosed herein, has severe or progressive genital HSV-related herpes infection.

For use in accordance with this aspect, the appropriate dosage is expected to vary depending on, for example, the mode of administration, and the nature and severity of the infection to be treated as well as the specific infection to be treated and is within the purview of the treating physician. Usually, an indicated administration dose may be in the range between about 0.1 to about 1000 μg/kg body weight.

A compound of the present disclosure may be administered by any conventional route, in particular: enterally, topically, orally, nasally, e.g., in the form of tablets or capsules, via suppositories, or parenterally. Suitable formulations and pharmaceutical compositions will include those formulated in a conventional manner using one or more physiologically acceptable carriers or excipients, and any of those known and commercially available and currently employed in the clinical setting. Thus, the compounds may be formulated for oral, buccal, topical, parenteral, rectal or transdermal administration or in a form suitable for administration by inhalation or insufflation (either orally or nasally).

Besides being useful for human treatment, the crystalline form of the present invention may be useful for veterinary treatment of companion animals, exotic animals and farm animals, including mammals, rodents, and the like. Conveniently, such animals include horses, dogs and cats.

### Combinations

Pritelivir Type 8 or pharmaceutical compositions comprising pritelivir Type 8 of the present invention may be administered alone as a sole therapy or can be administered in addition with one or more other substances and or treatments. Such conjoint treatment may be achieved by way of simultaneous, sequential or separate administration of the individual components of the treatment.

Also contemplated herein are methods that include administering a second active agent. For example, in addition to being infected with HSV, a subject or patient can further have HSV infection-related co-morbidities, i.e., diseases and other adverse health conditions associated with, exacerbated by, or precipitated by being infected with HSV. Contemplated herein are the crystalline Type 8 of pritelivir of the present invention or pharmaceutical compositions comprising the crystalline Type 8 of pritelivir of the present invention in combination with at least one other agent that has previously been shown to treat these HSV-infection-related conditions. Such conjoint treatment may be achieved independently (by way of simultaneous, sequential or separate administration of the individual components of the treatment) and/or via pharmaceutical compositions of the present invention that include a second active agent.

Therefore, provided herein is a method of treating HSV infection in a subject in need thereof, the method comprising administering a therapeutically effective amount of pritelivir Type 8 according to the present invention or a pharmaceutical composition comprising pritelivir Type 8 according to the present invention, to the subject and co-administering to the subject a therapeutically effective amount of an additional therapeutic agent.

In an embodiment, the additional therapeutic agent is selected from one or more of the following agents:
i. nucleoside polymerase inhibitors, such as acyclovir, valacyclovir, famciclovir, penciclovir, and ganciclovir;
ii. pyrophosphate polymerase inhibitors, such as foscarnet;
iii. saturated aliphatic alcohols, such as docosanol;
iv. agents such as idoxuridine, trifluridine and vidarabine;
v. a corticosteroid; and
vi. other helicase-primase inhibitors, such as amenamevir.

In some embodiments, pritelivir Type 8 or a pharmaceutical composition comprising pritelivir Type 8 may be administered as part of a combination therapy in conjunction with one or more antivirals, including nucleoside analogues such as acyclorvir, foscarnet, ganciclovir, or penciciovir or the respective prodrugs vaiaciciovir or famciclovir.

In some embodiments, the first and second amounts together comprise a pharmaceutically effective amount. The first amount, the second amount, or both may be the same, more, or less than effective amounts of each compound administered as monotherapies. Therapeutically effective amounts of a disclosed compound and antiviral may be co-administered to the subject, i.e., administered to the subject simultaneously or separately, in any given order and by the same or different routes of administration. In some instances, it may be advantageous to initiate administration of pritelivir first, for example one or more days or weeks prior to initiation of administration of the antiviral. Moreover, additional drugs may be given in conjunction with the above combination therapy.

### Process to Prepare Type 8

In one aspect, there is provided a process to prepare a crystalline form of pritelivir wherein the crystalline form is Type 8.

In one embodiment, the process to prepare Type 8 comprises the steps of:
a) providing a solution of pritelivir in a first solvent system;
b) adding a second solvent system to the solution from step a);
c) stirring the mixture obtained from step b) for at least 1 hour;
d) optionally, isolating the solids formed from step c); and
e) optionally, drying the solids isolated from step d).

In one embodiment, the first solvent system comprises a solvent wherein pritelivir has a solubility of at least 100 mg/mL at room temperature, such as at least 200, 300, 400 or 500 mg/mL. Suitably, the first solvent system comprises DMSO. Suitably, the first solvent system consists essentially of DMF. Suitably, the first solvent system consists of DMF.

Suitably, step a) is performed at room temperature.

Suitably, the second solvent system comprises a solvent wherein the pritelivir has a solubility of at less than 20 mg/mL at room temperature, such as less than 10 mg/mL. Suitably, the second solvent system comprises THF. Suitably, the second solvent system consists essentially of THF. Suitably, the second solvent system consists of THF.

Suitably, the first solvent system comprises DMSO and the second solvent system comprises THF.

Suitably, the second solvent system is an anti-solvent system.

Suitably, in step b) the second solvent system is added at room temperature.

Suitably, the second solvent system in step b) is added dropwise to the solution from step a).

Suitably, the addition of the second solvent system in step b) is over a time period of at least 10 minutes, such as at least 20 minutes, such as at least 25 minutes. Suitably, the addition of the second solvent in step b) is over a time period of about 30 minutes.

Suitably, the stirring in step c) is performed at room temperature.

Suitably, the stirring in step c) is performed for at least 1 hour, such as at least 2 hours, at least 4 hours, at least 8 hours, at least 12 hours, at least 18 hours or at least 22 hours.

Suitably, step d) comprises isolating the solids by centrifugation.

Suitably, step e) comprises drying the solids at a temperature greater than room temperature, such as greater than 30° C., or greater than 40° C. Suitably, step e) comprises drying the solids at a temperature of about 50° C.

The invention is illustrated below by the following non-limiting examples.

### EXAMPLES

The following abbreviations are used within this specification:
DMSO: Dimethyl sulfoxide
DSC: Differential scanning calorimetry
PXRD: Powder X-ray diffraction
RT: Room temperature
TGA: Thermogravimetric analysis
THF: Tetrahydrofuran
XRPD: Powder X-ray diffraction

### INSTRUMENTATION AND METHODS

Polarized-Light Microscopy (PLM): The photomicrographs were collected using Olympus BX53 polarized-light microscope. The sample was dispersed with methyl silicone oil on a glass slide.

Powder X-Ray Diffraction (PXRD): PXRD diffractograms were acquired on Rigaku Smartlab SE diffractometer using Cu, Kα, Kα1 (Å): 1.540598, Kα2 (Å): 1.544426 radiation with a Kα2: Kα1 intensity ratio of 0.50. X-Ray tube setting was 40 kV, 15 mA, Scan Mode 1D. The scan range (2 theta) was 3-40°, step size (2 theta) of 0.02° with scan speed (2 theta) of 10°/min.

Differential Scanning calorimetry (DSC): DSC was conducted with a TA Instruments DSC2500 differential scanning calorimeter using temperature ramp of room temperature to 300° C. with a heating rate of 10° C./min. The purge gas was N₂ (>99.999%). Punched aluminum pans were used.

Thermogravimetric Analysis (TGA): TGA thermograms were obtained with a TA Instrument TGA500 thermogravimetric analyzer using a temperature ramp of room temperature to 300° C. with a heating rate of 10° C./min. The purge gas was N₂ (>99.999%). Open aluminum pans were used.

### Example 1: Preparation and Characterization of Type 8

### Preparation of Type 8

pritelivir was charged into 4 mL glass vial, and 2 volumes of DMSO was added to obtain a clear solution with magnetic stirring at RT. The anti-solvent THF was dropwise added into the DMSO solution, and then the precipitation was gradually formed, and the suspension was kept stirring for 24 hours at RT. The solid was isolated by centrifugation and dried at 50° C., and then tested by XRPD, DSC and TGA.

### Characterization of Type 8

The characterization data for Type 8 is shown in FIGS. 1 and 2.

Type 8 was shown to be crystalline by XRPD (FIG. 1). DSC shows two small endothermic events with onset temperatures at ˜31° C. and 189° C., followed by a sharp endotherm having an onset temperature of 224° C. (FIG. 2). TGA shows a gradual 1.3% weight loss between 34 and 185° C.

### Example 2: Solubility Determination of Type 8

### Method

Sample preparation: excess Type 8 was added to the vehicle containing 0.9% NaCl and 0.5% polysorbate 80 to achieve ˜3 mg/mL suspension. The mixture was sonicated for 15 minutes, then agitated at 500 rpm at room temperature. At 24 hours, the mixture was filtrated, and filtrate was analyzed by HPLC to determine the solubility.

### HPLC Methodology:

Instrument: Agilent 1260
Column: ACE ultracore super C18 (100 mm*4.6 mm*2.5 μm) (P/N: CORE-25A-1046U)
Wavelength: 234 nm
Column Temperature: 20° C.
Column Flow: 1.0 mL/min
Acquisition time: 25 min
Injection volume: 5 μL
Retention time: ˜14 min
Mobile Phase A: 5 mM (NH₄)₂HPO₄ and 5 mM of NH₄H₂PO₄ in H₂O
Mobile Phase B: ACN

| | ACN:H₂O = 50:50 (v/v) | | |
|---|---|---|---|
| Needle Wash | Time(min) | A % | B % |
| Gradient Program | Initial | 90 | 10 |
| | 4 | 60 | 40 |
| | 11 | 60 | 40 |
| | 13 | 20 | 80 |
| | 20 | 20 | 80 |
| | 21 | 90 | 10 |
| | 25 | 90 | 10 |

### Results

The solubility of Type 8 at RT in 0.9% NaCl and 0.5% polysorbate 80 at 24 hours was around 70.7 ug/mL.

### Comparative Solubility Determination of Form C (Hemihydrate) as Disclosed in WO 2018/096170

### Method

The solubility of Form C (hemihydrate as disclosed in WO 2018/096170) was determined in the same media used to determine the solubility of Type 8 as described above.

Form C was prepared as follows:

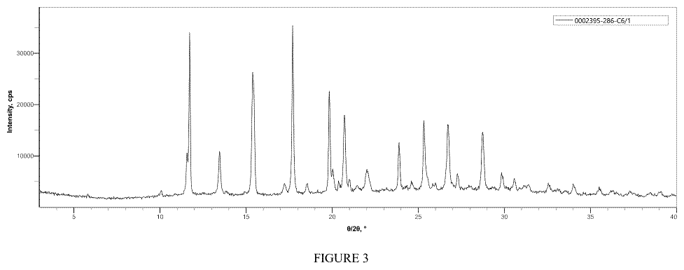

2-(4-(pyridin-2-yl)phenyl) acetic acid (38.40 g, 80%, 180.074 mmol, 1.00 eq) was added to DMF (385 mL). HOBT (17.38 g, 128.624 mmol, 0.71 eq) was added at 20° C., and then stirred for 10 minutes. 2-chloro-4-methylthiazole-5-sulfonamide (26.66 g, 128.624 mmol, 0.71 eq) and EDCI (27.12 g, 141.486 mmol, 0.79 eq) were added. The mixture was stirred for 36 hours at 20° C. under N₂. The mixture was added into water (780 mL) and stirred for 30 minutes. The solid was collected by filtration. The filter cake was washed with water twice (2×100 mL) and then dried under reduced pressure at 50° C. for 12 hours to give a white solid. Form C was obtained by slurrying this white solid in ethanol:water (v:v=1:1) followed by filtration at 50° C. Form C was confirmed to be crystalline by XRPD (FIG. 3).

Sample preparation: excess Form C was added to the vehicle containing 0.9% NaCl and 0.5% polysorbate 80. The mixture was sonicated for 15 minutes, then agitated at 500 rpm at room temperature. At 24 hours, the mixture was filtrated, and filtrate was analyzed by HPLC to determine the solubility.

### HPLC Methodology:

Instrument: Agilent 1260

Column: ACE ultracore super C18 (100 mm*4.6 mm*2.5 μm) (P/N: CORE-25A-1046U)

Wavelength: 234 nm

Column Temperature: 20° C.

Column Flow: 1.0 mL/min

Acquisition time: 25 min

Injection volume: 5 μL

Retention time: ˜14 min

Mobile Phase A: 5 mM (NH₄)₂HPO₄ and 5 mM of NH₄H₂PO₄ in H₂O

Mobile Phase B: ACN

| | ACN:H₂O = 50:50 (v/v) | | |
|---|---|---|---|
| Needle Wash | Time(min) | A % | B % |
| Gradient Program | Initial | 90 | 10 |
| | 4 | 60 | 40 |
| | 11 | 60 | 40 |
| | 13 | 20 | 80 |
| | 20 | 20 | 80 |
| | 21 | 90 | 10 |
| | 25 | 90 | 10 |

### Results

The solubility of Form C at RT in 0.9% NaCl and 0.5% polysorbate 80 at 24 hours was around 8.4 μg/mL, which is lower than that determined for Type 8 in the same media.

1. A crystalline form of pritelivir, wherein the crystalline form is Type 8 and is characterized by data selected from one or more of the following:
a) an X-ray powder diffraction (XRPD) pattern measured using Cu Ka (λ=1.5406 Å) comprising peaks at 2-theta values of 10.5 and 24.7°2θ±0.2°2θ; and
b) a DSC thermogram comprising an endothermic event with an onset temperature of 224° C.±2° C.

2. The crystalline form of claim 1 wherein the form is characterized by an XRPD pattern measured using Cu Ka (λ=1.5406 Å) comprising peaks at 2-theta values of 10.5 and 24.7°2θ±0.2°2θ.

3. The crystalline form of claim 1, wherein the form is characterized by an XRPD pattern measured using Cu Ka (λ=1.5406 Å) radiation comprising peaks at 2-theta values of 10.5 and 24.7°2θ±0.2°2θ and further comprising at least one, two, three or four specific peaks selected from peaks at 2-theta values of 17.4, 17.9, 23.0 and 23.3°2θ±0.2°2θ.

4. The crystalline form of claim 1, wherein the form is characterized by an XRPD pattern measured using Cu Ka (λ=1.5406 Å) radiation comprising peaks at 2-theta values of 10.5 and 24.7°2θ±0.2°2θ and further comprising peaks at 2-theta values of 17.4, 17.9, 23.0 and 23.3°2θ±0.2°2θ.

5. The crystalline form of claim 1, wherein the form is characterized by an XRPD pattern measured using Cu Ka (λ=1.5406 Å) radiation comprising at least two, five, ten, fifteen, twenty, twenty-five, thirty or thirty-five further peaks selected from the group consisting of the peaks in Table 1 in °2θ±0.2°2θ.

6. The crystalline form of claim 5 wherein the form is characterized by an XRPD pattern measured using Cu Ka (λ=1.5406 Å) radiation substantially the same as shown in FIG. 1.

7. The crystalline form of claim 1, wherein the form is characterized by a DSC thermogram comprising an endothermic event with an onset temperature of 224° C.±2° C.

8. The crystalline form of claim 1, wherein the form is characterized by a DSC thermogram substantially the same as FIG. 2.

9. The crystalline form of claim 1, wherein the form is substantially pure.

10. The crystalline form of claim 1, wherein the form comprises less than 2% by weight solvent and/or water.

11. A pharmaceutical composition comprising a crystalline form of pritelivir and a pharmaceutically acceptable carrier, diluent or excipient, wherein the crystalline form is Type 8 according to claim 1.

12. A method of treating a herpes virus (conveniently a HSV) infection in a subject in need thereof, the method comprising administering to the subject a therapeutically effective amount of a crystalline form of pritelivir or a pharmaceutical composition comprising the crystalline form of pritelivir, wherein the crystalline form is Type 8 according to claim 1.

13. (canceled)

14. A process to prepare a crystalline form of pritelivir, wherein the crystalline form is Type 8 and the process comprises the steps of:
a) providing a solution of pritelivir in a first solvent system;
b) adding a second solvent system to the solution from step a);
c) stirring the mixture from step b) for at least 1 hour;
d) optionally, isolating the solids formed from step c); and
e) optionally, drying the solids isolated from step d).

15. A crystalline form of pritelivir obtainable by the process according to claim 14.
